(19) United States
(12) Patent Application Publication
(10) Pub. No.: US 2021/0205023 A1
(43) Pub. Date: Jul. 8, 2021

(54) REAL-TIME LOCATION SYSTEM FOR MINIMUM LIGHT, PROBE OR LASER FIBER SURGICAL INTERVENTIONS FOR INTERNAL SURGERY

(71) Applicants: Joaquin Sabaris Vilas, Polinya Del Valles (ES); Sergio Mario Haimovich Segal, San Cugat Del Valles (ES)

(72) Inventors: Joaquin Sabaris Vilas, Polinya Del Valles (ES); Sergio Mario Haimovich Segal, San Cugat Del Valles (ES)

(21) Appl. No.: 17/056,456

(22) PCT Filed: May 16, 2019
(86) PCT No.: PCT/ES2019/070322

§ 371 Date: Nov. 18, 2020

(30) Foreign Application Priority Data
May 18, 2018 (ES) U201830723

(51) Int. Cl. A61B 34/20; A61B 1/303; A61B 18/22; A61B 8/08; A61B 8/00

(52) U.S. Cl. CPC A61B 34/20; A61B 1/303; A61B 18/22; A61B 2017/00199; A61B 8/483; A61B 8/54; A61B 8/466; A61B 8/0841

(57) ABSTRACT

Real-time localization system for minimal light surgical interventions, probe or fiber laser for internal surgery, composed of a probe or fiber optic laser, associated with the corresponding hysteroscope, an ultrasound machine with 3D technology and active image management, both associated with a control unit with a user interface and at least one screen for monitoring the location status of the optical fiber, for the emission of the laser and for the angle of incidence in the degree of temperature transfer to the myometrial wall, with the peculiarity that the ultrasound system has a system for guiding and monitoring its position on the skin layer of the patient, while the probe or optical fiber has a main fiber bundle for the transmission of the evaporation laser beam to a certain wavelength and at a certain power, modulated and controlled from the control unit, as well as a second beam of emission and reception bidirectional by rebound effect, pulsed laser for the practice of thermography, having provided that the control unit software includes means to obtain the positioning of the optical fiber based on the joint signals of the ultrasound machine, the internal probe and the system thermography associated with the second optical fiber bundle. Although the invention has been primarily intended to be applied in the field of myomatosis, the system of the invention is applicable to any type of minimal light surgical intervention in which probes or laser fibers are used.

### OBJECT OF THE INVENTION

The present invention relates to a real-time localization system for minimal light, probe or fiber laser surgeries in three spatial axes plus (internal) thermography for internal surgery.

The object of the invention is therefore to allow surgical interventions to be carried out in conditions of minimum light, with the greatest possible precision, based on the location in real time of both the instruments and the element to be intervened.

Although the invention has been primarily intended to be applied within the scope of myomatosis, the system of the invention is applicable to any type of minimal light surgical intervention in which probes or laser fibers are used.

### BACKGROUND OF THE INVENTION

Uterine myomatosis consists of the appearance of benign tumors, which are commonly referred to as: myomas, fibromas or uterine leiomyomas and which develop from the myometrium of the uterus, with different morphology and symptomatic or asymptomatic.

These can occur throughout the uterine cavity and are classified by their type and morphology, occurring in 1 out of 4 women of childbearing age, diagnosed between 30 and 40 years of age.

Its typology, in most cases, will mark its morphology and, at the same time, its location inside and outside the uterine cavity, being classified as follows:

### Subserous:

The most common, representing approximately 55% of the cases; growing towards the abdominal cavity and being able to take the place of adjacent organs from a certain development and size, although they are usually asymptomatic.

### Intramural:

Medium-frequent, in 40% of cases. Located between the thickness of the myometrium and proliferating in the central part of the myometrium, which increases the size of the uterus.

Submucosal (convertible into malignant):

As the less frequent (5-10%). Oriented in its vortex towards the uterine cavity, but instead, representing those with the greatest symptoms (hypermenorrhea) and subsequent iron deficiencies in addition to infertility.

In the surgical procedures described in the medical literature for their removal by hysteroscopy, using rigid laser capillaries at a certain wavelength, a series of complexities of approach to the instruments are presented, more or less marked by the size of the myoma and its uterine location; Mainly accused is the lack of precision in the input approaches of the laser light tip and the depth of action and the transmission of temperature towards the walls, which is why the patient has to be anesthetized or pauses of intervention have to be established.

Access for submucosal fibroids is vaginal, being so minimally invasive that in many cases the procedure can be performed in consultation and without anesthesia. These fibroids are divided into degrees depending on the penetration into the abdominal cavity, the GØ are 100% inside the cavity, G1 more than 50% in the cavity a small portion inside the wall, G2 mostly inside the wall and a small portion (<50%) within the endometrial cavity.

Intramural fibroids have an abdominal access requiring general anesthesia and an abdominal approach, either by laparoscopy or laparotomy.

It is in the case of G2 submucosal fibroids and intramural fibroids where we find orientation problems when introducing the laser, as we cannot control what is not seen. In this sense, the non-visible portion can vary in morphology, size and position.

To do this, and to date the location of the myoma is based on obtaining 3D tomographic images previously made, so that on said images trajectories for the probe or laser fiber are specified, with the problem that said images are not current, that is, they are not in real time, with the precision problems that this It can suppose, and that can cause perforations of the muscular wall, uncontrolled irradiations that cause an excess of heat in the area, etc.

### DESCRIPTION OF THE INVENTION

The recommended system acts as a locator and guide for the laser fiber or probe in the internal path through the uterus to the entrance to the myoma, thus obtaining in real time the image of the tumor and the depth reading necessary to proceed with the ablation of the mass by evaporation of the inner nucleus of each lobe of the myoma, in the necessary guarantees that avoid the perforation of the muscular wall and the controlled irradiation of the heat caused by the effect of the laser.

This means being able to intervene without the prior use of local anesthetics and the possibility of accessing the surgical field through the same light as the hysteroscope light, regardless of the previous dimensions of the tumor and correcting the current limitations of the field of view of the same in the magnitude of the scope of its coupled camera. It is also adjusted in cases in which there is more than one fibroid (bilobular fibroids), and in interventions of various kinds in which this same technique has the same advantages.

To this end, the system of the invention involves a series of electronic modules intercommunicated by means of an interpretation software that, in turn, acts as a user interface and a representation on the screen of the optical fiber. Both for the laser emission and for the angle of incidence in the degree of temperature transfer to the myometrial wall.

This makes it possible to solve the internal control of temperature transfer to the organ through internal thermography, with the delimitation of areas to establish pauses or greater cooling by recirculation of intrauterine fluid. These areas are adjustable in the thermograph reading circle.

Thus, the system of the invention makes it possible to locate the working laser probe in the intrauterine space by calculating its trajectory, in addition to giving interpretation to the inter-dimensioning of the cavities of the organ, thus establishing the delimitation of areas of heat transfer by the action of the laser towards adjacent tissues.

The invention represents an applied improvement derived from the current 3D ultrasound, which applies continuous readings of the apical slices to establish the recognition of the projected image in time, which is defined as the 4D echo, and at a higher sampling frequency (definition), such as 5D.

The invention extends its range of benefits to what could be called 6D, based on the addition of internal field thermography, modifying the dynamic response and resolution of the previous electronic readings of each processed image, making them closer to a new 6D ultrasound concept (5D+the reading captured from the thermography) with a calculation of 6 trajectory axes, which allows to expand the reading ranges and sampling frequency for each of the signals.

The use of this reading in real time, with the previous reference of the dimensioning of masses that marks the 3D echo or MRI before the surgical intervention, and that validates the actual size of the tumor mass, allows the establishment of different approaches to the same tumor and the alternative management or use of lower cost probes, such as lateral probes, regardless of the morphology and size of the myoma and without influencing the limitations of the Distal view of the cameras attached to the hysteroscope.

Consequently, the system of the invention provides for surface echo-guidance, through the combined use of an ultrasound machine with 3D technology and with active image management for 6D magnification, and a support that acts as a preliminary fixation of the piezoelectric crystal. of the ultrasound machine on a Cartesian fixation base in two-dimensional X and Y axes, with the addition of an angular module, valid to establish the initial reference points or zero reading offset (support fixed to the contour of the patient or external stand), an echo localization guide is established to detect and correct set-up lags that avoid anechoic or other intermediate lipid layer interferences. The probe acts as an internal emitter for its location.

On the other hand, the calculation algorithms will be applied according to the previous records (Cartesian) to be able to extrapolate a scaled 3D image of the already known ultrasound technique, except for the superposition details that save the present invention and that make it possible to establish a complete visualization of the area of action and guided echo in real time of the probe on its way to the tumor mass.

This characteristic also benefits the use of lateral probes that are cheaper and that will allow a fractal scraping of the interior of the myoma or misshapen mass; both in the circular ones and in the bilobar dysmorphic ones.

This interface allows the user to control and correct trajectories and improve approximations and specify areas of laser firing and expected heat transfer.

Another novel feature of the invention is the fact of being able to establish self-adjustments by tumor typology, for greater ease of handling and guidance of the system.

Regarding the optical fiber used, it is based on a known fiber for the transmission of the main beam of the evaporation laser (FO) at a certain wavelength and at a certain power, modulated and controlled from a control unit, but with the addition of an enveloping upper layer through which the bidirectional emission and reception, due to the rebound effect, of the pulsed laser from the source for the thermography calculation.

Thus, in the main core of the optical fiber, the laser acts as a thermal scalpel to ablate the tumor by evaporation and removal of tissue by recirculating the widening fluid of the uterine cavity by hysteroscopy (being the one that supports the greatest load of power), while the outer layer of shorter length and with a variable angle of attack, acts as a means of transferring the laser emission linked to the thermographic measurement of the external walls of the tumor.

From this structuring, and together with the initial set-up, the echo-guidance of the probe and the reception of reading of its positioning with respect to the uterine interspacing and the myoma tumor chamber is allowed, as a field of intervention, so that it is not necessary to specify previous trajectories on the first 3D tomographic image (initial reference). This determines the reading of the dimensioning and the approach angle and the depth of action in real time, during the intervention.

For all these reasons, the direct approach is facilitated regardless of tumor morphology, their size (exceeding 4 cm in diameter) and availability of external resources, being able to be of application in the ablation by evaporation of intrauterine fibroids and others of diverse nature, in intra-extra-hospital settings.

### DESCRIPTION OF THE DRAWINGS

To complement the description that is going to be made below and with the aim of helping to better understand the characteristics of the invention, according to a preferred example of a practical embodiment thereof, a set of drawings is attached as an integral part of said description. where, for illustrative and non-limiting purposes, the following has been represented:

FIG. 1.—Shows a schematic representation of the system of the invention for the intervention or ablation of an intramural myoma, all carried out in accordance with the object of the present invention.

FIG. 2.—Shows a representation with the essential elements that participate in the system of the invention.

FIG. 3.—Shows the whole 6D intrauterine localization system.

FIG. 4.—Shows a view of the fibroids and offset points.

FIG. 5.—Shows the offset gain points.

FIG. 6.—Finally shows an abdominal image with offsets.

### PREFERRED EMBODIMENT OF THE INVENTION

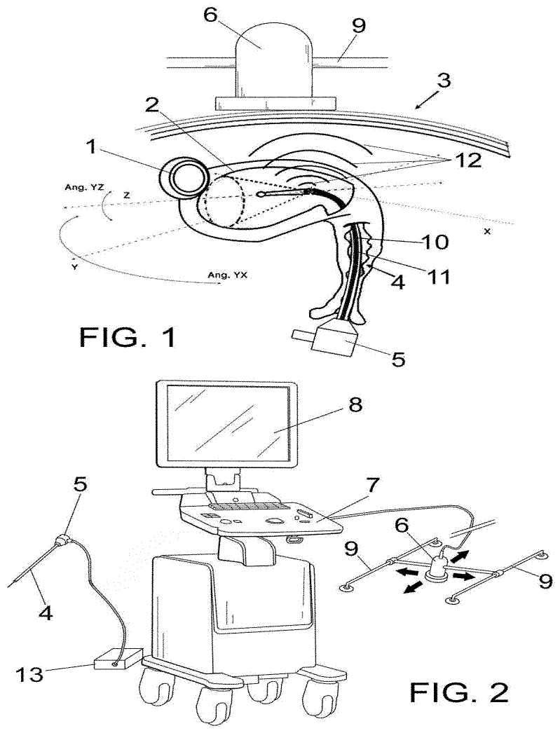

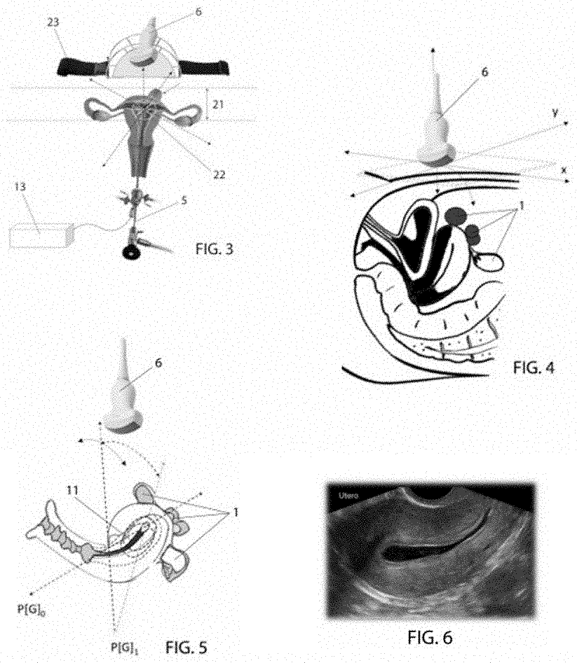

In FIG. 1, and merely by way of example, an intramural myoma (1) is shown, formed between the inner wall of the uterus (2) and the dermal and epiploic/mesenteric layers (3) of the patient.

Well, according to the invention, the system of the invention is based on the simultaneous use of a series of devices, all of them managed by software and controlled from a user interface with its corresponding microprocessor and control unit (7) in which a screen (8) represents the location status of the optical fiber (4), both for the laser emission and for the angle of incidence in the degree of temperature transfer to the myometrial wall.

Consequently, among said devices there is a laser probe or optical fiber (4) associated with the corresponding hysteroscope (5), as well as an ultrasound machine (6) with 3D technology and active image management.

More specifically, the ultrasound machine (6) is placed on the skin layer (3) of the patient through a guidance system (9) duly monitored by the control unit (7), guidance system (9) in the that participate in slide-guides in two-dimensional X, Y, and angled axes, valid to establish the initial reference points or zero reading offset, defining a Cartesian guide to detect and correct the set-up lags that avoid anechoic interferences or others of intermediate lipid layer.

For its part, the probe or optical fiber (4), in addition to the function of transmitting element of the main beam of the evaporation laser, acts as a surface emitter for its location.

To do this, said optical fiber (4) has a main fiber beam (10) for transmitting the evaporation laser beam (FO) at a certain wavelength and at a certain power, modulated and controlled from the control unit (7), with the particularity that it includes a second beam (10) as an envelope, through which the bidirectional emission and reception, due to the rebound effect, of a pulsed laser from the source for the calculation of thermography, passes.

Consequently, in the main core of the optical fiber, the laser acts as a thermal scalpel to ablate the tumor by evaporation and removal of tissue by recirculation of the widening fluid of the uterine cavity by hysteroscopy (being the one that supports the greatest load of power), while the outer layer of shorter length and with a variable angle of attack, acts as a means of transferring the laser emission linked to the thermographic measurement of the external walls of the tumor.

According to FIG. 1, for the oriented control of the cutting probe or fiber optic (4), in its intrauterine location, the “XYZ” axes are defined in addition to the angular “ZY” and ZX, and for the rotation the axis “φ” with respect to the pelvic base, being on the axis “Z” in particular, the one that marks the depth of the introduced section of the workpiece with respect to the point “0” and the thermal reading the transfer. A technical detail is added to the probe in this section that acts as a reference for the positioning system and that validates the dynamic response of the proposed echolocation.

This determines six 6 positioning variables+application thermography.

From this structuring, and together with the initial set-up, the echo-guidance of the probe and the reception of reading of its positioning with respect to the uterine interspacing and the myoma tumor chamber is allowed, as a field of intervention, so that it is not necessary to specify previous trajectories on the first 3D tomographic image (initial reference).

1. Real-time localization system for minimal light surgery, probe or fiber laser for internal surgery, characterized in that it involves a laser probe or optical fiber (4), associated with the corresponding hysteroscope (5), as well such as an ultrasound machine (6) with 3D technology and active image management, both associated with a control unit (7) with a user interface and at least one screen (8) for monitoring the location status of the optical fiber (4), both for the laser emission and for the angle of incidence in the degree of temperature transfer to the myometrial wall, with the particularity that the ultrasound machine (6) has a guidance system (9) and position monitoring of the same on the dermal layer of the patient, while the probe or optical fiber (4) has a main fiber beam (10) for the transmission of the evaporation laser beam at a certain wavelength and at a certain power, modulated and controlled from the control unit (7), as well as a second beam (10) of bidirectional emission and reception by rebound effect, of pulsed laser for the practice of thermography, having provided that the software of the control unit (7) include means for obtaining the positioning of the optical fiber (4) based on the joint signals of the ultrasound machine (6), the internal probe (11) and the thermography system associated with the second beam (10) of the optical fiber (4).
